SEQUENCE LISTING (5 sequences)

SEQ ID NO: 1 - DNA, 51 bp, Homo sapiens
attccagatagagctaaaactgaagytttccttatagagatttatcctagt

SEQ ID NO: 2 - DNA, 27 bp, Artificial Sequence
  Description of Artificial Sequence Synthetic primer
tctgtgatagagtggcattagaaattc

SEQ ID NO: 3 - DNA, 25 bp, Artificial Sequence
  Description of Artificial Sequence Synthetic primer
ccccgcaaactaactaggataaatc

SEQ ID NO: 4 - DNA, 17 bp, Artificial Sequence
  Description of Artificial Sequence Synthetic probe
ctaaaactgaagctttc

SEQ ID NO: 5 - DNA, 20 bp, Artificial Sequence
  Description of Artificial Sequence Synthetic probe
aactgaagttttccttatag

(19) United States
(12) Patent Application Publication
(10) Pub. No.: US 2023/0100349 A1
(43) Pub. Date: Mar. 30, 2023

(54) PHASE-ISOLATED WATER-IN-OIL TRANSPARENT MACROEMULSION AND APPLICATION THEREOF

(71) Applicant: Enumerix, Inc., Palo Alto, CA (US)

(72) Inventors: Peng FEI, Hubei (CN); Peiyu LIAO, Beijing (CN); Mengcheng JIANG, Beijing (CN); Fangli ZHANG, Beijing (CN); Jun NIE, Hubei (CN)

(21) Appl. No.: 17/286,421

(22) PCT Filed: Oct. 18, 2019
(86) PCT No.: PCT/CN2019/111938

§ 371 Date: Mar. 8, 2022

(30) Foreign Application Priority Data
Oct. 19, 2018 (CN) 201811221827.X

(51) Int. Cl. C12Q 1/6851

(52) U.S. Cl. CPC C12Q 1/6851; C12Q 1/6848

(57) ABSTRACT

The present disclosure relates to a combined reagent for preparing a water-in-oil transparent emulsion and droplets formed by the reagent. The present disclosure also relates to a use of the droplets in digital polymerase chain reaction.

### CROSS-REFERENCES TO RELATED APPLICATIONS

The present application claims the benefit of the Chinese patent application No. 201811221827. X filed on Oct. 19, 2018 entitled “A phase-isolated water-in-oil transparent macroemulsion and use thereof”, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on May 26, 2022, is named 56396-706_831_SL.txt and is 1,358 bytes in size.

### TECHNICAL FIELD

The invention belongs to a technical field of digital polymerase chain reaction detection, and more specifically, relates to a phase-isolated water-in-oil transparent macroemulsion and application thereof. The phase-isolated water-in-oil transparent macroemulsion is especially a stable transparent macroemulsion of which water and oil are isolated in two phases, and an application of the transparent macroemulsion system can enable digital quantitative detection, and a corresponding detection method can especially enable high-specificity digital closed nucleic acid quantitative detection.

### BACKGROUND

The digital polymerase chain reaction (dPCR) is currently the most sensitive and accurate nucleic acid quantification method in the world. The digital detection herein means that a solution or suspension containing analyte, for example biological macromolecules such as nucleic acids, proteins, or bacteria, cells, is divided into independent reaction spaces which are isolated from each other but have the same volume, so that the analytes are randomly dispersed in these micro-reactors for independent detection reactions. When the individual number of analytes is equivalent to or smaller than the number of reaction partitions, some of the reaction partitions show positive signals and the rest show negative signals (because there is no analyte in this partition). Once the number of positive partitions is counted, concentration of the analyte can be calculated by using the Poisson distribution statistical model. Although the polymerase chain reaction is a common detection reaction, since the reaction is a thermal cycling reaction, temperature changing and controlling equipment with higher precision is required; while thermostatic reactions such as loop-mediated amplification, rolling circle amplification, recombinant polymerase amplification, and multiple displacement amplification are also used in many scenarios due to sensitive response, simple operation, and low equipment requirements. Since digital detection can be quantified at the level of a single nucleic acid molecule, single protein molecule or single cell, bacteria, it can be said that it is the most accurate quantitative method and can distinguish the difference between single bases in the nucleic acid sequence, which has obvious advantages in many scenarios.

In order to increase the reaction throughput of the digital polymerase chain reaction, it is necessary to set up multiple independent reaction partitions. At present, there are a variety of methods that can form independent reaction partitions, among which there are “hard divisions” with the use of micro-processing devices such as microfluidic chips to achieve micron-level micro-wells, and “soft divisions” of uniform droplets formed with water-in-oil. The latter has gradually occupied a larger market share in scientific research and practical applications due to its simplicity and ease of operation and low cost. In the “soft divisions”, emulsion droplets are one of the powerful tools that are practical and rapidly developing in the field of chemical biology and have many applications in the fields of life sciences and materials. The size of the emulsion droplets is usually between several micrometers and several hundreds of micrometers, and they stably exist in the oil phase liquid under the action of specific surfactants. Due to current method limitations and technical conventions, etc., the emulsion droplets in scientific research and production are generally between 1 micron and 300 microns. Emulsion droplets can uniformly disperse the sample droplets (mostly aqueous solutions) into multiple divisions with nearly the same volume, and the isolated divisions can form independent partitions, which can greatly increase the reaction throughput, and can be used for the synthesis of microscale and numerous crystalline particles, polymer pellets, etc. The size of the formed solid particles is similar, and the synthesis process is easy to regulate and control.

The article [1] published by Schulman et al. in June 1961 and the textbook “Colloidal and Surface Chemistry” [2] written by SHEN Zhong et al. describe the classification of emulsions, in which the emulsions are classified into emulsion and microemulsion according to the particle size of the emulsion. “Emulsion” may be used interchangeably with “macroemulsion.” The present disclosure will continue to use the definition of the macroemulsion. Microemulsion generally refers to a liquid dispersion system in which the size of dispersed particles is small and their diameter is less than 0.1 μm. Since the size of the particles is smaller than the wavelength of visible light, the transmitted light is less interfered with by refraction and scattering, and the microemulsion is usually present as a stable system that is transparent or slightly semitransparent in appearance. However, the macroemulsion generally has a larger particle size of 0.1 μm or more, which will cause the passing light to scatter and refract under natural conditions, and therefore the macroemulsion generally appears opaque milky white. Microemulsion usually contains at least four components, water, oil, surfactants and co-surfactants, wherein the co-surfactants are mostly alcohols, especially polyhydroxy compounds. The macroemulsion contains no co-surfactants but contains the remaining three components. The natural transparent property of microemulsion is favored in cosmetics. Many patents use the transparent property to improve the appearance of products. For example, in the references [4-7], fatty alcohols or polyhydroxy substances are added as co-emulsifiers, such as glycol, esters of sorbic acid, PEG and PPG; these substances often have HBL values of more than IO to ensure good solubility in water andoil. Although the microemulsion is stable as a whole in appearance and has no change to the naked eye, the inside of the microemulsion is a large number of flexible films formed by emulsifiers and co-emulsifiers with HBL value of more than 10. Due to the presence of co-emulsifiers, the formation and dissolution of the film are in the dynamic change all the time, and meanwhile the substances in the water and oil exchange with this layer of film at all times. References [8-9] mentioned that transparent microemulsion is used to achieve chemical reactions (transesterification reaction) between two immiscible liquid phases, the transparent emulsion here is not used to achieve better optical properties or product appearance but used to provide a great contact opportunity for the two immiscible liquid phases, thereby greatly improving the reaction rate of the chemical process. Microemulsion systems such as those in references [4-7] are often used in the field of cosmetics, mainly used to improve product appearance and user experience, in which the size of micelles is uncontrollable, uneven, or are not micelles at all but are many molecular films, which cannot be subjected to thermal cycles, and cannot achieve phase separation since the droplet permeability is high (the solute can be exchanged between water and oil phases). In addition, many components (e.g., polyhydroxy substances such as fatty alcohols, and glycerol) that inhibit the activity of bio-enzymes are added to the microemulsion, and therefore the microemulsion is not suitable for biochemical reactions, let alone digital reactions. However, there is obvious phase separation in the macroemulsion system, that is, most of the substances in the water and oil phases will not enter the other phase: this is due to the stable droplets formed in the macroemulsion, in which the interface formed by the ordered arrangement of surfactants has certain rigidity and insulation properties. Due to the phase separation characteristics of the macroemulsion, the macroemulsion can form multiple independent reaction spaces, and has better thermal stability, and the droplet size is suitable for the digital reaction system. Compared with the microemulsion, the macroemulsion system is more suitable for the digital reaction.

The phase-isolated macroemulsion droplets can greatly improve the accuracy and resolution of the detection and quantification methods based on limiting dilution strategies such as digital polymerase chain reaction. The currently common digital quantitative techniques such as digital bacterial count, digital cell count, and digital polymerase chain reaction are all based on the uniform division characteristic of macroemulsion droplets. The strategy is divided into three steps: sample dropletization partitioning, signal amplification reaction, and count processing. There are usually two methods for counting and processing fluorescence droplets: the droplets pass through the microfluidic channel one by one and are counted sequentially at the fluorescence detection point, and the droplets are spread on a flat surface or rotating cylindrical surface and information such as the position and number of fluorescence droplets is obtained by a fluorescence imaging method. However, there are deficiencies in the above two methods, i.e., one-by-one detection and counting, and planar photography method. The droplets in one-by-one detection and counting method are in a flowing state, a flow rate stabilizing the emulsion is needed, and thus additional microfluidic control is required. For emulsions with high viscosity or dense droplets, it is also necessary to add diluent oil to space the droplets before the emulsion enters into the detection point. The planar photography method can only photograph up to three layers of droplets, and it is almost impossible to achieve the imaging of droplets in deeper layers without special treatment due to the refraction. In addition, both counting methods require imaging in a specific container, which will definitely involve the transfer of amplified products. This approach will most likely contaminate subsequent experiments, greatly increasing the probability of false positives in subsequent experiments.

Therefore, there is an urgent need for a method for in situ closed imaging detection of macroemulsion droplets in deeper layers.

### REFERENCES

1. J. K. Schulman & J. B. Montage, FORMATION OF MICROEMULSIONS BY AMINO ALKYL ALCOHOLS, Annals New York Academy of Sciences, vol. 92, issue 2, 366-371, June, 1961.
2. SHEN Zhong, ZHAO Zhenguo, KANG Wanli, “Colloid and Surface Chemistry”, Chemical Industry Press, 2011 edition.
3. J. Z. Sun, M. C. E. Erickson, & J. W. Parr, Refractive Index Matching and Clear Emulsions, J. Cosmet. Sci., 56, 256-265, July & August, 2005.
4. U.S. Pat. No. 5,925,338, CLEAR ANTIPERSPRIRANT OR DEODORANT GEL COMPOSITION WITH VOLATILE LINEAR SILICONE TO REDUCE STAINING, Nancy M. Karassik, et al, 1999.
5. U.S. Pat. No. 6,403,069, HIGH OIL CLEAR EMULSION WITH ELASTOMER, Suman Chopra, et al, 2002
6. U.S. Pat. No. 6,387,357, HIGH OIL CLEAR EMULSION WITH DIENE ELASTOMER, Suman Chopra, et al, 2002.

### 7. TRANSPARENT OIL-IN-WATER EMULSION, Michel F. Mercier, et al, 2009.

8. U.S. Pat. No. 3,480,616, ESTERIFICATION OF POLYHYDRIC COMPOUNDS IN THE PRESENCE OF TRANSPARENT EMULSIFYING AGENT, Lloyd I. Osipow, 1969.
9. U.S. Pat. No. 3,644,333, TRANSETERIFICATION IN THE PRESENCE OF A TRANSPARENT EMULSION, Lloyd I. Osipow, 1972.
10. Chinese Patent, Application No. CN 201610409019.0, Publication No. CN106076443A, HUANG Yanyi et al., 2016.
11. Chinese Patent, Publication No. CN 106053346 B, “A Light Sheet Microscopic Imaging Device”, FEI Peng et al., 2016.

### SUMMARY

In view of the above deficiencies or improvement needs of the prior art, the object of the present disclosure is to provide a phase-isolated water-in-oil transparent macroemulsion and application thereof. Through using a specific transparent macroemulsion formula, a preparation method of the corresponding transparent macroemulsion droplets, and an imaging detection method of the transparent macroemulsion droplets, and by controlling the composition of both the water and liquid phases of the water-in-oil macroemulsion used for phase isolation, especially by adjusting the refractive index of the water phase in the macroemulsion droplets, the macroemulsion droplets are transparent, so that light can pass through the transparent droplets in the shallow layer to reach the droplets in the deep layer, thereby enabling the in-situ closed imaging detection of the macroemulsion droplets in the deep layer. In the present disclosure, by controlling the water phase and liquid phase of the water-in-oil macroemulsion used for phase isolation (for example, the specific component types and corresponding proportions of the oil phase system and the water phase system respectively), the obtained phase-isolated water-in-oil macroemulsion realizes for the first time that the transparent droplets have many advantageous properties at the same time: (1) controllable and uniform size; (2) transparent, achieving centimeter-level penetration in the ultraviolet-visible light band; (3) obvious phase isolation, the solvents in the water phase and the oil phase are not easily exchanged, and the reaction is closed; (4) the activity of biological macromolecules such as polymerase was not affected; (5) mechanically and thermally stable, and the droplets will not be broken due to cyclic heating and movement. Due to these advantages, it is possible to achieve the in-situ closed imaging detection of the macroemulsion droplets in the deep layer by this system, and this system has a wide range of application prospects.

In one aspect, the present disclosure provides a combination reagent for preparing a water-in-oil transparent emulsion, which includes: a water phase reagent in which a refractive index enhancer is dissolved; and an oil phase reagent in which a surfactant is dissolved; wherein when an optical detection is performed, the water-in-oil droplets formed by the water phase reagent and the oil phase reagent have an imaging depth of at least 300 microns.

In some embodiments, an absolute value of a difference between refractive indexes of the water phase reagent and the oil phase reagent is not more than 0.1. In some embodiments, the absolute value of the difference between the refractive indexes of the water phase reagent and the oil phase reagent is not more than 0.01.

In some embodiments, a mass percentage of the refractive index enhancer in the water phase reagent is not less than 20%. In some embodiments, the mass percentage of the refractive index enhancer in the water phase reagent is from 25% to 40%. In some embodiments, the refractive index enhancer is at least one selected from the group consisting of inorganic salt, monosaccharide, disaccharide, polysaccharide derivative, amino acid, polar organic compound, dimethyl sulfoxide, formamide, tetramethylammonium chloride and bovine serum albumin. In some embodiments, the polar organic compound is at least one selected from the group consisting of acetylcholine, choline, betaine, and ceramide. In some embodiments, the amino acid is at least one selected from the group consisting of glycine, arginine, threonine, and lysine.

In some embodiments, the oil phase reagent comprises at least one matrix selected from the group consisting of fluorocarbon oil, hydrocarbon-based oil, silicone-based oil and derivatives thereof. In some embodiments, an HBL value of the surfactant in the oil phase is not more than 8. In some embodiments, a mass percentage of the surfactant in the oil phase reagent is from 0.1% to 20%. In some embodiments, the mass percentage of the surfactant in the oil phase reagent is from 2% to 10%.In some embodiments, the surfactant is at least one selected from the group consisting of Dow Corning® 5200 Formulation Aid, Dow Corning® 9011 Silicone Elastomer Blend, Dow Corning® 5225C Formulation Aid, Dow Corning® BY 11-030, Dow Corning® BY 25-337, Dow Corning® ES-5612 Formulation Aid, Dow Coming® FZ-2233, Dow Corning® ES-5226 DM Formulation Aid, Dow Corning® ES-5227 DM Formulation Aid, MASSOCARE SIL series, KF-6017P, KF-6028P, Chemsil K-12, Abil® EM 97 and SilCare Silicone® WSI.

Another aspect of the disclosure provides a transparent emulsion, which comprises: a water phase in which a refractive index enhancer is dissolved, an oil phase in which a surfactant is dissolved, and an analyte; wherein the water phase forms discrete droplets, the oil phase forms a continuous phase, the analyte is in the discrete droplets of the water phase, and when an optical detection is performed, the droplets have an imaging depth of at least 300 microns.

In some embodiments, a volume percentage of the water phase in the transparent emulsion is from 5% to 90%. In some embodiments, the volume percentage of the water phase in the transparent emulsion is from 10% to 30%.

In some embodiments, the analyte is selected from nucleic acid, protein, bioactive molecule, bacteria, and cell. In some embodiments, the nucleic acid is a nucleic acid molecule that has been amplified. In some embodiments, the nucleic acid is a nucleic acid molecule that has not been amplified.

Another aspect of the disclosure provides a droplet for providing an independent micro-encapsulation environment, which is obtained by emulsifying and dispersing the combined reagent described in the present disclosure. In some embodiments, an average diameter of a water phase droplet in the droplet is not less than 0.2 μm. In some embodiments, when an optical detection is performed, the droplet has an imaging depth of at least 300 microns. In some embodiments, the droplet provides an independent micro-encapsulation environment for a digital polymerase chain reaction.

Another aspect of the disclosure provides a use of the droplets of the disclosure in an in-situ closed imaging detection is provided. In some embodiments, use of the droplets described in the present disclosure in a digital polymerase chain reaction is provided.

Another aspect of the disclosure provides a method for preparing water-in-oil droplets for a digital polymerase chain reaction, which comprises: preparing a water phase reagent and an oil phase reagent, respectively, wherein the water phase reagent is dissolved with a refractive index enhancer, the oil phase reagent is dissolved with a surfactant, and an absolute value of a difference between refractive indexes of the water phase reagent and the oil phase reagent is not more than 0.1; and performing emulsification and dispersion treatment to cause the water phase to enter into the oil phase to form the droplets.

In some embodiments, the method further comprises mixing the water phase reagent with an analyte. In some embodiments, the water phase reagent is mixed with the analyte before the emulsification and dispersion treatment.

In some embodiments, the emulsification and dispersion treatment is selected from vibration emulsification treatment, microfluidic cross co-flow treatment, dropletization treatment with microfluidic T-type flow channel method, and centrifugal droplet emulsification treatment.

Another aspect of the disclosure provides a method of digital polymerase chain reaction, which comprises: (1) dispersing an analyte in a water phase reagent in which a refractive index enhancer is dissolved; (2) contacting the water phase solvent with the oil phase reagent in which a surfactant is dissolved to form a water-in-oil emulsion, and wherein the analyte is in the formed water phase droplets; (3) amplifying the analyte in the droplets; and (4) performing an optical detection process on the droplets.

In some embodiments, the amplification is selected from polymerase chain reaction, multiple displacement amplification reaction, recombinase polymerase isothermal amplification reaction, loop-mediated isothermal amplification reaction, or rolling circle amplification reaction.

In some embodiments, the optical detection is light sheet scanning imaging. In some embodiments, the method has sensitivity of a single base.

Another aspect of the disclosure provides a phase-isolated water-in-oil transparent macroemulsion, which is characterized in that the transparent macroemulsion is obtained by emulsifying and dispersing a water phase and an oil phase, wherein an average diameter of water phase droplets dispersed in the oil phase is not less than 0.2 μm; and a refractive index enhancer is also dissolved in the water phase, and a mass percentage of the refractive index enhancer component in the entire water phase is not less than 20%; a surfactant with HBL value of not more than 8 is also dissolved in the oil phase; in addition, an absolute value of the difference between refractive indexes of the water phase and the oil phase is not more than 0.1.

As a further preference of the present disclosure, the mass percentage of the refractive index enhancer component in the entire water phase is from 25% to 40%; the absolute value of the difference between the refractive indexes of the water phase and the oil phase is not more than 0.01.

A volume percentage of the water phase in the entire water-in-oil transparent macroemulsion is from 5% to 90%, more preferably from 10% to 30%.

As a further preference of the present disclosure, an oil phase matrix in the oil phase is a fluorocarbon oil, hydrocarbon-based oil, or silicone-based oil, preferably a silicone-based oil with a viscosity of not less than 0.5 cSt, preferably, the viscosity of the silicone-based oil is not higher than 10 cSt, and the oil phase matrix is more preferably a silicone-based oil with a viscosity of 1 cSt.

As a further preference of the present disclosure, a mass percentage of the surfactant in the entire oil phase is from 0.1% to 20%, preferably from 2% to 10%;

The oil phase matrix in the oil phase is specifically a silicone-based oil, and the surfactant is a silicone-based surfactant, preferably Dow Coming® 5200 Formulation Aid, Dow Coming® 9011 Silicone Elastomer Blend, Dow Corning® 5225C Formulation Aid, Dow Corning® BY 11-030, Dow Corning® BY 25-337, Dow Corning® ES-5612 Formulation Aid, Dow Corning® FZ-2233, Dow Corning® ES-5226 DM Formulation Aid, Dow Corning® ES-5227 DM Formulation Aid, MASSOCARE SIL series, KF-6017P, KF-6028P, Chemsil K-12, Abil® EM 97, or SilCare Silicone® WSI.

As a further preference of the present disclosure, the phase-isolated water-in-oil transparent macroemulsion contains no glycol, polyethylene glycol, and small molecular fatty alcohols with a relative molecular mass of not more than 1000, and a surfactant with an HBL value of more than 8.

As a further preference of the present disclosure, the refractive index enhancer is selected from at least one of inorganic salt, monosaccharide, disaccharide, polysaccharide derivative, amino acid, polar organic compound, dimethyl sulfoxide, formamide, tetramethylammonium chloride and bovine serum albumin.

Preferably, the inorganic salt is a chloride or sulfate of potassium, calcium, sodium, magnesium, zinc, manganese, or iron element; the monosaccharide is glucose, fructose or sorbose; the disaccharide is sucrose; the polysaccharide derivative is cellulose acetate, hydroxypropyl methylcellulose or sodium carboxymethyl cellulose; the amino acid is glycine, arginine, threonine or lysine; the polar organic compound is acetylcholine, choline, betaine or ceramide.

The refractive index enhancer is preferably betaine, glycine, arginine, threonine, and lysine.

According to another aspect of the present disclosure, the present disclosure provides a combined reagent for forming a water-in-oil transparent macroemulsion, characterized in that the combined reagent comprises both a water phase combined reagent and an oil phase combined reagent, wherein, the water phase combined reagent comprises a water solvent and a refractive index enhancer that can be dissolved in water, and a mass percentage of the refractive index enhancer in the entire water phase combined reagent is not less than 20%; the water phase combined reagent includes an oil phase matrix and a surfactant with an HBL value of not more than 8; the water phase combined reagent is used for mixing to form a water phase, the oil phase combined reagent is used for mixing to form an oil phase, an absolute value of the difference between refractive indexes of the water phase and the oil phase is not more than 0.1, and both the water phase and the oil phase are used for further emulsification and dispersion to obtain the transparent water-in-oil macroemulsion.

According to another aspect of the present disclosure, the present disclosure provides a method for preparing phase-isolated water-in-oil transparent macroemulsion droplets, characterized in that the phase-isolated water-in-oil transparent macroemulsion droplets belong to the aforementioned phase isolated water-in-oil transparent macroemulsion, and the method includes the following steps: (1) preparing a water phase and an oil phase, respectively, both of which are used to form the above-mentioned phase-isolated water-in-oil transparent macroemulsion; (2) performing emulsification and dispersion treatment so that the water phase enters into the oil phase to form transparent macroemulsion droplets.

As a further preference of the present disclosure, in the step (2), the emulsification and dispersion treatment is specifically vibration emulsification treatment, microfluidic cross co-flow treatment, dropletization treatment with microfluidic T-type flow channel method, or centrifugal droplet emulsification treatment, preferably centrifugal droplet emulsification treatment.

According to another aspect of the present disclosure, the present disclosure provides the application of the above-mentioned phase-isolated water-in-oil transparent macroemulsion in providing an independent micro-encapsulation environment;

the phase-isolated water-in-oil transparent macroemulsion preferably provides an independent micro-encapsulation environment for the high specificity digital polymerase chain reaction; preferably, the high specificity digital polymerase chain reaction is used for detection;

preferably, the purpose of providing the independent micro-encapsulation environment is for digital bacterial counting detection, or for the detection or quantitative analysis of protein or nucleic acid, or for protein crystallization, processing or observation, or for the study of molecular communication of bacteria or cells in a three-dimensional environment.

According to another aspect of the present disclosure, the present disclosure provides a detection method based on a specificity digital polymerase chain reaction, which is characterized in that it includes the following steps: (1) forming the macroemulsion droplets according to the above method of preparing the phase-isolated water-in-oil transparent macroemulsion droplets, when preparing the water phase therein, adding nucleic acid, protein molecule, bioactive molecule, bacteria or cell having a target concentration; (2) carrying out a specificity digital polymerase chain reaction on the macroemulsion droplets obtained in step (1); (3) performing an optical detection on the macroemulsion droplets obtained from the reaction in step (2) to obtain the number and/or concentration of nucleic acid, protein molecule, bioactive molecule, bacteria or cell having the target concentration; preferably, in the step (2), the specificity digital polymerase chain reaction is polymerase chain reaction, multiple displacement amplification reaction, recombinase polymerase isothermal amplification reaction, loop-mediated isothermal amplification reaction or rolling circle amplification reaction; in the step (3), the optical detection is preferably light sheet scanning imaging.

In the present disclosure, by controlling the specific composition of the water phase and the oil phase in the macroemulsion (and the corresponding specific formulation of the combined reagent used to form a water-in-oil transparent macroemulsion), especially by adjusting the refractive index of the water phase in the macroemulsion droplets to obtain transparent macroemulsion droplets, the light can pass through the transparent droplets in the shallow layer to reach the droplets in the deep layer, thereby enabling the in-situ closed imaging detection of the macroemulsion droplets in the deep layer. The macroemulsion contains two phases, i.e., the water phase and the oil phase. Under normal circumstances, most of the macroemulsions are opaque, and present milky white or off-white. This is because the refractive indexes of the oil and water phases are different, at the same time, the size of the emulsion droplets is larger than one-fourth of the wavelength of the light, the specific surface area is large, and the two-phase interface exists everywhere inside the macroemulsion. The light undergoes refraction and scattering when it contacts the two-phase interface, sometimes the light is absorbed inside the droplets and cannot pass through in a straight line. However, when the refractive indexes of the water and oil phases are the same or similar, the two-phase interface disappears, and the light can pass through in a straight line, and the emulsion becomes transparent. The degree of transparency of the emulsion changes with the difference between the refractive indexes of the oil and water phases. If one wants to obtain a transparent emulsion, the difference between the refractive indexes of the oil phase and the water phase is within ±0.1 (at this time, it can be considered that the refractive indexes of the water and oil phases are similar), preferably within ±0.01. In addition, when the phase-isolated water-in-oil transparent macroemulsion droplets are used in specific applications, nucleic acids, protein molecules, bioactive molecules, bacteria or cells, etc. having the target concentration can be added to the water phase, and the emulsion droplets at this time may also become opaque.

TABLE 1

| Liquid (20° C.) | Refractive index |
|---|---|
| Water | 1.330 |
| Perfluoroalkanes | 1.238 to 1.303 |
| (Fluorinert ™ | |
| series products) | |
| Silicone-based oil | 1.375 to 1.430 |
| Benzene, Toluene | 1.501, 1.497 |
| n-octane, isooctane | 1.398, 1.391 |
| C8-C12 fatty ester | 1.43 to 1.46 |

The refractive index enhancer used in the present disclosure is used to adjust the refractive index of the water phase, so that the refractive index of the water phase meets the requirements of matching the refractive index of the oil phase, and the refractive index enhancer is needed to be able to dissolve in water under natural conditions (from 20 to 40° C.); the refractive index enhancer can be selected from various biological reaction additives which are commonly used.

The refractive indexes of water and some common oil phases are shown in Table 1. It can be seen from Table I that the refractive index of the water phase is less than that of most oil phases. To increase the refractive index of the water phase to be the same or similar as that of the oil phase, the refractive index enhancer needs to be added. To increase the refractive index of the water phase while maintaining a large rigidity of the droplet interface and high isolation degree (low permeability) to form a closed independent reaction chamber, the refractive index enhancer is required to not disrupt the phase isolation effect. Even the silane with the smallest difference compared to water has a refractive index difference of more than 0.4, if refractive index matching is desired, a molar-level enhancer needs to be added. If it is an organic small molecule with a small molecular weight, the addition amount should usually be more than 2 moles, as this concentration is sufficient to interfere with most biological reactions. In particular, the present disclosure uses specific types of water-phase refractive index enhancers (including inorganic salts, such as chloride or sulfate of potassium, calcium, sodium, magnesium, zinc, manganese, and iron; monosaccharides or disaccharides such as glucose, sucrose, sorbose, and fructose; polysaccharide derivatives such as cellulose acetate, hydroxypropyl methylcellulose, sodium carboxymethyl cellulose; amino acids such as arginine, threonine, and lysine; strong polar organic compounds such as acetylcholine, choline, betaine, and ceramide; commonly used biological reaction additives such as dimethyl sulfoxide, formamide, tetramethylammonium chloride and bovine serum albumin) that neither disrupt the phase isolation effect nor interfere with the biochemical reactions, the refractive index of the water-phase can be increased to match that of the silicone-based oil by preferably adding these specific water-phase refractive index enhancers, thereby obtaining transparent macroemulsion droplets.

In particular, when biochemical reactions are needed to be carried out in a droplet system, the refractive index enhancer is preferably betaine. Betaine is an additive commonly used in biochemical reactions, which is known to help DNA molecules to eliminate the influence of secondary structure and the influence of GC base ratio on melting temperature. This molecule is an amphoteric organic salt, has little effect on the ionic strength of the water solution, and little interference with biological enzymes, and has the highest addition concentration among the most commonly used additive in nucleic acid amplification reactions and protein stabilization reaction solutions. The generally commonly used concentration is I to 2 moles per liter, which is much higher than the 0.2 moles per liter of other additives. We found that a larger allowable concentration of betaine provides a flexible space for the adjustment of refractive index, so that the refractive index of the water phase reaction solutions is sufficiently improved, and betaine can become the main refractive index adjustment substance of the water phase liquid for biochemical reactions. Our experiments have found that in the bulk reaction, the maximum addition concentration of betaine is generally less than 3 moles per liter, mostly around 2 moles per liter, and the concentration of higher than 3 moles per liter will result in biological enzymes (such as polymerases commonly used in nucleic acid amplification reactions) becoming ineffective, resulting in failure of the reaction. However, in a system with an extremely small volume of macroemulsion droplets, when the addition amount of betaine reaches 4 moles per liter, it still does not significantly affect the progress of the polymerase chain reaction. The reason is that the larger specific surface area of the small system can increase the probability of molecular collision. On the other hand, the amplified product is restricted in a microenvironment, making the local environment concentration several orders of magnitude higher than that in the bulk reaction, which can overcome the negative impact caused by decreased enzyme activity. In addition, small-molecular organic amphoteric ion salts, such as amino acids, which have a similar refractive index enhancement effect as betaine, also have a higher addition concentration in the emulsion system and can also be used as a suitable refractive index enhancer.

In addition, the addition concentration of the refractive index enhancer is determined according to the refractive index requirements. The higher the addition concentration, the more the refractive index of the water phase increases, the smaller the difference between the refractive indexes of the water phase and the oil phase, and the more transparent the emulsion. In addition to using a single refractive index enhancer component, the present disclosure can use multiple refractive index enhancers at the same time. In the present disclosure, the mass percentage of the refractive index enhancer component in the entire water phase is preferably from 25% to 40%, and the desired addition concentration can be calculated according to the refractive index increment brought by the unit molar concentration; for refractive index enhancers compatible with most of the biochemical reactions, if the refractive index is needed to be increased by 0.1, the concentration of the refractive index enhancer in the water phase will be increased by I mole per liter to 5 moles per liter. The addition of the refractive index enhancer will not interfere with the phase isolation performance of the droplets, and there is no need to add surface active substances to the water phase of the emulsion formulation.

In order to match the refractive index of the oil phase with that of the water phase more easily, it is preferable to choose an oil phase with a refractive index close to that of the water phase as much as possible. The oil phase of the macroemulsion can be a silicone-based oil or a derivative thereof.

In the present disclosure, silicone-based oil is the first choice as the oil phase of the emulsion. Silicone-based oil is a liquid polymer of polydimethylsiloxane, and its polymerization degree can be high or low. In silicone oil products, the higher the polymerization degree of silicone oil, the longer the molecular chain, the higher refractive index of the silicone oil, and the greater the viscosity of the silicone oil. However, the change rate of refractive index is much smaller than that of viscosity, and therefore the larger selection range of silicone oil viscosity can adapt it to a larger range of microfluidic rheological requirements. Using silicone-based oil as the oil phase matrix has a stronger ability to dissolve other substances than perfluoroalkanes and is easier to adjust the refractive index; and it is also easier to form an emulsion system that is stable and compatible with the biological reactions compared to octane or alkanes with a similar number of carbon atoms, the benefits are obvious.

In the present disclosure, the volume percentage of the water phase in the entire water-in-oil macroemulsion is preferably controlled to be 5% to 90% (more preferably 10% to 30%), so that the thermal and mechanical stability of the macroemulsion can be ensured.

In order to obtain a stable transparent macroemulsion, a suitable hydrophobic and lipophilic surfactant is also required in addition to the matched refractive index of the oil and water phases. The present disclosure uses a surfactant with an HBL value of not more than 8 in the oil phase. Surfactants dissolved in silicone oil can be silicone-based, which contributes to stabilize the silicone oil-water interface. Silicone-based surfactants have a wide range of chemical modifications, which can adapt to the compatibility requirements of different chemical and biological systems. The surfactant can also be a fluorocarbon-based or a hydrocarbon-based, or derivatives of polydimethylsiloxane. When the subsequent biochemical reactions need to be carried out in the emulsion droplets, the requirements of the subsequent reactions on the surfactants need to be considered, such as thermal stability under multiple thermal cycles, biocompatibility, nonfoaming, no significant protein adsorption, and no heavy metals, no biological residues such as nucleic acids or proteins. The present disclosure preferably uses silicon-oxygen chain surfactants using PEG (polyethylene glycol), PPG (polypropylene glycol) as the hydrophilic group (e.g., Dow Coming® 5200 Formulation Aid), and silicon-oxygen chain surfactants such as SilCare Silicone® WSI using glycerin ester as the hydrophilic group. In the present disclosure, the mass percentage of the surfactant in the entire oil phase is adjusted to be 0.1% to 20% (preferably 2% to 10%), so that the advantage of enhancing the stability of the oil-water interface can be brought.

Unlike the transparent microemulsion, the transparent macroemulsion of the present disclosure is unnecessary to contain small molecular fatty alcohols, because the addition of fatty alcohols produces a microemulsion system with a particle size of less than 0.2 μm, and at the same time affects the emulsion isolation performance, biocompatibility and subsequent detections. To perform digital detection in droplets, the medium is required to has mild property, moderate acidity and alkalinity, and its ionic strength does not interference with the biochemical reactions, and at the same time, the medium is required to ensure that the droplet size is uniform, and it does not fuse or break during temperature rise or repeated high-low temperature conversions. At the same time, it has a certain resistance to mechanical shock. The addition of fatty alcohol will interfere with the enzymatic reaction, and its surface activity is prone to cause the uneven size of the microemulsion droplets and damage the thermal stability at the same time.

In the present disclosure, different oil phases can be selected in terms of different dropletization methods. For example, when centrifugal droplet emulsification is used, short-chain low-viscosity silicone oil without chemical modifications is preferred as the oil phase, which can avoid the generation of debris when the droplets hit the oil surface. The specific viscosity is not higher than I0 cSt, but considering that the lower the viscosity, the more flammable it is, from a viewpoint of safety, the viscosity should be around I cSt. If the microfluidic chip is used to generate droplets, the viscosity of the silicone oil can be adjusted (since the silicone oil products generally do not indicate polymerization degree, the viscosity can be used to characterize the polymer chain length) to achieve the design purposes such as adjusting the size of the droplets. Silicone oil with fatty chain modifications generally has a higher viscosity, which is conducive to the production of small droplets, but when the viscosity is too large, the difficulty in controlling the flow rate will be increased. In order to increase the solubility of gas in the oil phase, meet the requirements of gas exchange between the water phase and the external environment, and at the same time reduce the extraction of small molecules in the water phase that are slightly soluble in oil by the oil phase, the fluoro-modified or other halo-modified silanes can be used. For example, a mixture of multiple miscible silanes can be used as the oil phase to meet various needs.

The phase-isolated water-in-oil transparent macroemulsion of the present disclosure is particularly applicable to high-specificity digital polymerase chain reactions and detection, and other biochemical reactions that require a separate micro-encapsulation environment (considering the size of biomolecules, the average diameter of the water phase droplets in the phase-isolated water-in-oil macroemulsion droplets is not less than 0.2 μm; the maximum upper limit of the average diameter can be adjusted according to the actual situation, for example, the average diameter does not exceed 200 μm). The present disclosure also provides a method for carrying out a highly specific digital polymerase chain reaction with a transparent emulsion, and the detection can be achieved by optical detection means.

The optical detection method of the transparent macroemulsion droplets can be selected according to the degree of transparency and the desired imaging depth, which can be an existing imaging method in the prior art, such as light sheet scanning imaging (e.g., Reference [11]), wide field scanning, brightfield imaging, confocal imaging, and the light sheet scanning imaging is preferred. Compared with other imaging methods, a deeper imaging depth can be obtained by using the light sheet scanning imaging. The types of detection can also include fluorescence, absorption and turbidity, or a combination thereof, among which the fluorescence can also be polychromatic. The detection signal acquisition can be terminal signal acquisition or real-time signal acquisition.

In general, the present disclosure has achieved the following technical effects:

First, the transparent macroemulsion formula of the present disclosure uses a surfactant with an HBL value of less than 8, without adding co-surfactants such as small molecular fatty alcohols, and uses a refractive index modifier with good biocompatibility to form a water-in-oil phase-isolated macroemulsion system with a particle diameter not less than 0.2 μm, its internal droplet morphology and phase isolation performance have good mechanical and thermal stability, and is suitable for biological reactions, which provides a foundation for enabling the biochemical applications of isolated micro-encapsulation environments, especially the application in highly specific digital polymerase chain reactions.

Second, the present disclosure preferably adopts the betaine or the amino acid as the refractive index enhancer to ensure that the transparent droplet component is compatible with subsequent biochemical reactions under the condition of adjusting the refractive index.

Third, the present disclosure can enable in-situ closed imaging through the transparentizing of droplets, eliminating the complex equipment and product transferring steps required by the conventional detection methods, improving sample reading speed and throughput, increasing user operation convenience, and reducing sample contamination.

Fourth, in the present disclosure, the transparent macroemulsion formula (such as controlling the respective specific component types and corresponding proportions of the oil phase system and the water phase system) is combined with the centrifugal droplet emulsification, which can produce a large number of transparent macroemulsion droplets with good uniformity in a short time; the droplets' are generated in the reaction vessel by the method of the centrifugal droplet emulsification, and the reaction can be carried out without sample transferring, which is suitable for biological reactions in multiple parallel independent reaction systems; the transparent macroemulsion droplets can ensure imaging in deep depth, and after reaction, the imaging detection can be directly carried out without sample transferring and opening a cover.

Fifth, the method of polymerase chain reaction is carried out by usmg the transparent macroemulsion droplets in the present disclosure, and it is proved that the detection of single-base sensitivity can be enabled by the present disclosure through a digital PCR reaction.

Sixth, in the present disclosure, the transparent macroemulsion droplets is combined with the light sheet imaging, and the deep droplet imaging can still be achieved at a depth of more than 300 microns from the imaging side of the sample, the closed in-situ imaging of the deep droplets can be performed without opening the container cover, greatly reducing the probability of product contamination.

### BRIEF DESCRIPTION OF THE DRAWINGS

FIG. I is a schematic diagram of the transparency of the macroemulsion droplets after changing the concentration of the refractive index enhancer. Due to the change in the concentration of the refractive index enhancer, the macroemulsion droplets will exhibit different transparency. In the figure, the concentration of the refractive index enhancer increases from left to right, the transparency first increases and then decreases, and it is most transparent at the appropriate concentration (the third from the right).

FIG. 2 is a diagram showing the fluorescence droplets after amplification by the digital polymerase chain reaction. It can be seen from the figure that after adding more than 3 mol/L of the refractive index enhancer, the effect of the digital polymerase chain reaction will not be affected. The fluorescence signal has a sharp contrast in the positive droplets, indicating that there is no significant exchange of substances among the droplets.

FIG. 3 is a diagram showing light sheet chromatographic results obtained from polymerase chain reaction solutions with betaine solution with a series of gradient concentration (the addition amount of 5 mol/liter of betaine stock solution is linearly increased from top to bottom). On the rightmost is a histogram of the refractive index of each group, and the horizontal line represents the refractive index of the emulsified oil.

FIG. 4 is a diagram showing the imaging results of the macroemulsions under different refractive index matching conditions (betaine with gradient concentrations) and different illumination conditions. Among them, the first row: transmission; the second row: wide-field fluorescence imaging; and the third row: light sheet illumination fluorescence imaging.

FIG. 5 is a diagram showing results of the digital polymerase chain reactions with index matching. When the light sheet scanning imaging is used, light sheet fluorescent photographs of the transparent emulsion droplets are transparentized at different depths. This figure is spliced with 9 thumbnails with a sequence number from I to 9, and the sequence number from I to 9 represents the fluorescence images of the excitation planes of different depths. It can be seen that although the signal-to-noise ratio of the final image is slightly reduced, it does not affect the counting of the fluorescence droplets number.

FIG. 6 is a diagram showing results of single-base mutation detection through a digital polymerase chain reaction using transparent macroemulsion droplets. This figure is spliced with 3 thumbnails with a sequence number from I to 3, wherein thumbnail numbered I is obtained by superimposing the signals of thumbnail numbered 2 and thumbnail numbered 3, the thumbnail numbered 2 and the thumbnail numbered 3 are fluorescence images of the same sample at the same layer, wherein the thumbnail numbered 2 is the fluorescence signal of the 488 nm channel, and the thumbnail numbered 3 is the fluorescence signal of 532 nm. Obvious fluorescence enhancement appears in some of the droplets. It can be seen that although the concentration of the betaine solution added as the refractive index enhancer is as high as 3. ISM, polymerase chain reaction still occurs, which indicates that the refractive index enhancer does not reduce the specificity of the polymerase chain reaction and has the capability of enabling DNA detection and quantification with single base differences. In this experiment, two probes are used to detect one gene site, and there is only one base difference between the two probes. At the same time, two fluorescent molecules are used to correspond to two genotypes, respectively. The sample used is DNA of a heterozygous individual, and therefore both probes will show fluorescent signals. Since there is only one DNA molecule in each droplet in most cases, if this method is sensitive enough, only one signal will appear in a droplet; when two signals appear, it means that this method cannot distinguish the difference of a single base, and the sensitivity is not enough. It can be seen that the positions of the bright spots in the thumbnail numbered 2 and the thumbnail numbered 3 are different, and only a single signal is generated in the droplet, which indicates that the method is extremely sensitive and can effectively distinguish two different bases at the same site.

### DETAILED DESCRIPTION OF THE PRESENT DISCLOSURE

In order to clearly describe the objectives, technical solutions, and advantages of the present disclosure, the present disclosure will be described in further detail below in conjunction with the accompanying drawings and embodiments. It should be understood that the specific embodiments described herein are only used to explain the present disclosure instead of limiting the protection scope of the present disclosure. In addition, the technical features involved in the embodiments of the present disclosure as described below can be combined with each other so long as they do not constitute a conflict with each other.

The formulation of the transparent macroemulsion in the present disclosure is as follows: the macroemulsion contains a water phase and an oil phase, the water phase can account for 5% to 90% (preferably 10% to 30%) of the volume of the emulsion, and a refractive index enhancer is added to the water phase, wherein the enhancer can account for more than 20% of a total mass fraction of the water phase, and a surfactant with an HBL value of 8 or less is added to the oil phase, the refractive indexes of the water phase and the oil phase are the same or similar, and the droplet diameter after emulsification is 0.2 μm or more. The difference between the refractive indexes of the oil phase and the water phase can be controlled within ±0.1, preferably within ±0.01, to obtain a transparent emulsion. In order to ensure the thermal stability and mechanical stability of the macroemulsion, the ratio of the water phase in the emulsion is not too high or too low, and the volume ratio is generally 5% to 90%, preferably 10% to 30%.

Suitable water-phase refractive index enhancers that neither disrupt the phase isolation effect nor interfere with the biochemical reactions include inorganic salts, such as chloride or sulfate of potassium, calcium, sodium, magnesium, zinc, manganese, and iron; monosaccharides or disaccharides such as glucose, sucrose, sorbose, and fructose; polysaccharide derivatives such as cellulose acetate, hydroxypropyl methylcellulose, sodium carboxymethyl cellulose; amino acids such as arginine, threonine, and lysine; strong polar organic compounds such as acetylcholine, choline, betaine, and ceramide; commonly used biological reaction additives such as dimethyl sulfoxide, formamide, tetramethylammonium chloride and bovine serum albumin. The refractive index of the water phase can be increased to be consistent with that of the silicone oil by adding the above-mentioned water phase refractive index enhancer, thereby obtaining transparent macroemulsion droplets. The addition of the refractive index enhancer will not interfere with the phase isolation performance of the droplets, and there is no need to add surface active substances to the water phase of the emulsion formulation.

The oil phase of the macroemulsion can be silicone oil or derivatives thereof. As the degree of polymerization changes, its viscosity can range from 0.5 cSt to tens of millions of cSt. Silicone oil can be 317667 silicone oil or 378321 silicone oil (Sigma), and low viscosity oils such as Gelest DMS-T01 and DMS-T0I.5. Many properties of the silicone oil, such as refractive index, dissolution rate to certain solutes, wetting ability, density and viscosity, can be adjusted through chemical modifications. The modification on the siloxane skeleton can be halogen atoms such as fluorine and chlorine; linear or branched aliphatic groups such as ethyl, propyl, isopropyl, butyl, isobutyl, pentyl, octyl; linear or branched halogenated aliphatic groups; aromatic groups such as phenyl, fluorophenyl, benzyl and halogenated benzyl; and polar groups such as oligo-polyethylene glycol group, oligo-polyglycerol group, N-pyridylpropyl, tetrahydrofuranol propyl, and butylcyano group.

In order to obtain a stable transparent macroemulsion, in addition to the refractive index matching of the oil and water phases, a suitable hydrophobic and lipophilic surfactant is also preferable. The present disclosure uses a surfactant with an HBL value of not more than 8 in the oil phase. Surfactants dissolved in silicone oil can be silicone-based, also a fluorocarbon-based or a hydrocarbon-based, or a derivative of polydimethylsiloxane. When the subsequent biochemical reactions need to be carried out in the emulsion droplets, the requirements of the subsequent reaction on the surfactants need to be considered, such as thermal stability under multiple thermal cycles, biocompatibility, nonfoaming, no significant protein adsorption, and no heavy metals, and no biological residues such as nucleic acids or proteins. The present disclosure preferably uses silicon-oxygen chain surfactants using PEG (polyethylene glycol), PPG (polypropylene glycol) as the hydrophilic group (e.g., Dow Corning® 5200 Formulation Aid), and silicon-oxygen chain surfactants such as SilCare Silicone® WSI using glycerin ester as the hydrophilic group. In the present disclosure, the mass percentage of the surfactant in the entire oil phase is adjusted to be 0.1% to 20% (preferably 2% to 10%).

In the method for preparing transparent macroemulsion droplets of the present disclosure, the transparent macroemulsion droplets are stable spherical particles formed by emulsifying and dispersing the above water phase liquid in the oil phase. The preparation method generally includes the following steps: (1) preparing the water phase (containing a refractive index enhancer, etc.) and the oil phase (a lipophilic surfactant) liquid, respectively, and (2) emulsifying and dispersing to form the transparent macroemulsion droplets.

The method for emulsifying and dispersing the water phase into the oil phase to form droplets can be vibration emulsification, microfluidic cross co-flow method or T-type flow channel dropletization method or the centrifugal droplet emulsification method in the prior art (for example, Reference [10]), with these methods, droplets with adjustable diameter and good uniformity can be obtained.

In addition, different oil phases can be selected in terms of different dropletization methods. When centrifugal droplet emulsification is used, short-chain low-viscosity silicone oil without chemical modifications is preferred as the oil phase, which can avoid the generation of debris when the droplets hit the oil surface. The specific viscosity is not higher than IO cSt, but considering that the lower the viscosity, the more flammable it is, from a viewpoint of safety, the viscosity is to be about I cSt. If the microfluidic chip is used to generate droplets, the viscosity of the silicone oil can be adjusted (the viscosity can be used to characterize the polymer chain length) to achieve the design purposes such as adjusting the size of the droplets. Silicone oil with fatty chain modifications generally has a higher viscosity, which is conducive to the production of small droplets, but when the viscosity is too large, the difficulty in controlling the flow rate will be increased. In order to increase the solubility of gas in the oil phase, meet the requirements of gas exchange between the water phase and the external environment, and at the same time reduce the extraction of small molecules in the water phase that are slightly soluble in oil by the oil phase, the fluoro-modified or other halo-modified silanes can be used. In order to meet various needs, a mixture of multiple miscible silanes can be used as the oil phase.

The present disclosure also provides an application of the macroemulsion with the above formula or the emulsion droplets prepared by the above method for preparing the macroemulsion droplets in digital reaction and detection, wherein the digital reaction is for example bacteria counting, detection and quantification of protein and nucleic acid, protein crystallization, processing, observation, the study of molecular communication of bacteria or cells in a three-dimensional environment, or other biochemical reactions that require a separate micro-encapsulation environment.

The digital polymerase chain reaction is currently the most sensitive nucleic acid detection method. Due to the use of limiting dilution strategy, it is called “digital” detection. The limiting dilution strategy here refers to diluting and dispersing the analyte to be quantitatively detected (many are biological macromolecules such as DNAs, RNAs and other nucleic acids and proteins, or dispersed cells, viruses, and bacteria) to the same detection environment independent of each other, so that the number of analyte (the number of molecules in DNAs, RNAs and proteins, the number of cells, the number of viruses or the number of bacteria) does not exceed the number of the division reactions too much (preferably 3 times or less), and then, the concentration of the analyte is obtained by counting the number of positive divisions in each division reaction and using the Poisson distribution formula. The detection and quantitative method using this strategy has a resolution at the molecular level and is widely used in biomedicine, and new technologies about this principle are also being widely studied. The transparent emulsion droplets according to the present disclosure are particularly suitable for highly specificity digital polymerase chain reaction and detection.

In the present disclosure, the method of using a transparent macroemulsion to perform a highly specificity digital polymerase chain reaction can include the following steps: (1) preparing a transparent macroemulsion formula containing a water phase and an oil phase, wherein the water phase contains, for example, nucleic acid, (2) dropletizing the reaction water phase solution to obtain the transparent macroemulsion droplets, so that there is 0 or 1 nucleic acid molecule in most of the droplets, (3) performing polymerase chain reaction on the transparent macroemulsion droplets, and (4) performing optical detection on the transparent macroemulsion droplets obtained from the reaction.

After dropletizing the emulsion containing the nucleic acid sample, some of the droplets contain nucleic acid molecules, and some do not. In order to ensure the confidence of the Poisson distribution correction, it is preferable that the number of the nucleic acid molecules does not exceed the total number of the droplets, so that there is 0 or 1 nucleic acid molecule in most of the droplets. After the droplet undergoes 30 to 50 rounds of thermal cycling or homothermal nucleic acid replication, the nucleic acid molecules will exhibit exponential amplification. The amplified nucleic acid will generate a sufficiently strong fluorescent signal in the droplet containing the nucleic acid molecule by utilizing a suitable fluorescent signal generation method, and the concentration of the nucleic acid in the sample can be obtained by counting the fluorescent droplets, and the accuracy of the result thereof is at a molecular level.

The high specificity digital polymerase chain reaction is performed usmg conventional methods, and after the reaction is completed, the transparent macroemulsion droplets obtained from the reaction are subjected to optical detection. The optical detection method of the transparent macroemulsion droplets can be selected according to the degree of transparency and the desired imaging depth. The optical detection method can be a light sheet scanning imaging (e.g., Reference [11], wide field scanning, brightfield imaging, confocal imaging, where light sheet scanning imaging is preferred. Compared with other imaging methods, a deeper imaging depth can be obtained by using the light sheet scanning imaging. The types of detection can also include fluorescence, absorption, and turbidity or a combination thereof, among which the fluorescence can also be polychromatic. A detection signal acquisition can be terminal signal acquisition or real-time signal acquisition.

The following are specific examples:

### EXAMPLE 1

In order to verify the appropriate concentration of the refractive index enhancer, the Gelest DMS-TOI.5 silicone oil and the surfactant Dow Coming ES5612 were formulated at a mass ratio of 19:1, mixed uniformly and centrifuged at 20,000 rcf for 10 minutes to obtain the supernatant for the emulsified oil in the next step. Betaine in the water phase is a refractive index enhancer. A volume of water phase in each sample was 20 μL, and 240 μL of the above-mentioned oil was added to the system. FIG. 1 shows from left to right the addition of 0.5, 1.0, 1.5, 2.0, 2.5, 3.0, 3.5, and 4.0 moles per liter of refractive index enhancer (the concentration here refers to the concentration in the finally formed water-oil mixture), respectively. It can be seen from FIG. 1 that as the concentration of the refractive index enhancer increases, the transparency first increases and then decreases, and it is most transparent when the concentration of the refractive index enhancer is 3.0 moles per liter (i.e., the third from the right in FIG. 1).

### EXAMPLE 2

The difference from Example 1 is that glycine was used as the refractive index enhancer. The glycine solutions of different concentrations (0.5, 1, and 1.5 mol/L) were centrifuged to form macroemulsion droplets. As the concentration of glycine increases, the transparency first increases and then decreases, and it is most transparent when the concentration of the refractive index enhancer is 2.8 moles per liter.

### EXAMPLE 3

In this example, the digital polymerase chain reaction of the transparent macroemulsion droplets was included, and after the reaction was completed, the macroemulsion droplets were subjected to light sheet scanning imaging detection.

TaqMan® MGB (Applied Biosystem™) probe was used to detect the single-base mutations in the genome. There was only one base difference in a single base mutation, which is the most demanding in nucleic acid detection. There is a mutation on chromosome 8 in the genome of the tested volunteer, its SNP number is rs10092491, and the mutation sequence is:

(SEQ ID NO: 1)
ATTCCAGATAGAGCTAAAACTGAAG[C/T]TTTCCTTATAGAGATTTAT
CCTAGT

### 1. Primers and Probes for Detection

| | | 20XMixed |
|---|---|---|
| | Sequence | solution |
| Forward | 5′-TCTGTGATAGAGTGGCATTAGAAAT | 18 μM |
| primer | TC-3′ (SEQ ID NO: 2) | |
| Reverse | 5′-CCCCGCAAACTAACTAGGATAAAT | 18 μM |
| primer | C-3′ (SEQ ID NO: 3) | |
| FAM- | (FAM)-5′-CTAAAACTGAAGCTTTC-3′- | 5 μM |
| probe | (MGBNFQ) (SEQ ID NO: 4) | |
| HEX- | (HEX)-5′-AACTGAAGTTTTCCTTAT | 5 μM |
| probe | AG-3′-(MGBNFQ) (SEQ ID NO: 5) | |

The above-mentioned oligonucleotides were prepared into a 20× Mixed solution according to the concentration in the third column of the above table.

### 2. Preparation of polymerase Chain Reaction Solution

| | Concentration | Final | |
|---|---|---|---|
| | before | concentration | Addition |
| Components | dilution | after dilution | volume |
| I0X buffer-Mg* | 10X | IX | 10 μl |
| MgCh * | 50 mM | 4 mM | 8 μl |
| Betaine solution | SM | 3.15M | 63 μl |
| dNTP | 10 mM, each | 400 nM, each | 4 μl |
| PlatinumTaq ® | | | 1.5 μl |
| 20X Mixed | 20X | IX | 5 μl |
| solution | | | |
| DNA sample to | | | 5 μl |
| be tested | | | |
| Water | | | 3.5 μl |

*All are incidental by the PlatinumTaq ® products.

### 3. Formulation of Emulsified Oil

The Gelest DMS-T0I.5 silicone oil and the surfactant Dow Corning 5612 were formulated at a mass ratio of 19:1, mixed uniformly and centrifuged at 20,000 ref for 10 minutes to obtain the supernatant for the emulsified oil in the next step.

### 4. Generation of Centrifugal Droplets

The droplets were generated by using the methods as described in the prior art, such as in the Chinese Patent (Application No. CN201610409019.0, i.e., Reference [10]). The dropletization of a transparent emulsion was carried out by the emulsification method of centrifugal droplets in the present disclosure (see related prior art for details), the general procedures include: in a centrifuge, the water phase liquid was placed above a glass plate, and the glass plate had several small holes with the same size and a diameter of several microns. Under the action of centrifugal force, the water phase liquid passed through the small holes to form droplets with the same size, and the droplets then entered into the oil phase below to be stabilized by the surfactant dissolved in the oil phase to form a stable uniform emulsion, and droplets with different diameters can be obtained by adjusting the diameter of the small holes and the rotation speed of the centrifuge. In order to facilitate subsequent reactions and observations, the diameter of the droplets that can be generated is from 30 to 120 microns. In specific practice, due to many considerations during the detection process, the diameter of the selected droplets was 48 microns.

A 37-well, 6 μm microchannel array orifice plate was used in the present disclosure, and 16 μ1 of the formulated polymerase chain reaction solution was added to complex of the microchannel array plate and the collection device. The collection device was a 200 μL PCR tube, the PCR tube contained 240 μL of the above emulsified oil, centrifugal speed was 15,000 ref, centrifugal time was 4 minutes, and about 440,000 transparent droplets with a diameter of 41 microns were generated. (Note: The 200 μL PCR tube mentioned here refers to the specifications of the centrifuge tube, the actual volume of such PCR centrifuge tube is about 300 μL, however, the general addition amount in biological reaction is not more than 200 μL)

### 5. Thermal Cycling

The above droplets were placed in a thermal cycler and heated according to the procedures in the table below.

| | | Heated | |
|---|---|---|---|
| | | cover | |
| Heated | | before | |
| cover | 105° C. | cycling | start |
| Step 1 | Standing still | 25° C. | 120 s |
| Step 2 | Enzyme thermal | 95° C. | 120 s |
| | activation | | |
| Step 3 | Thermal cycling | 40 rounds | |
| Step 3.1 | Denaturation | 92° C. | 15 s |
| Step 3.2 | Annealing | 58° C. | 30 s |
| Step 4 | Cryopreservation | 4° C. | Continuing |

Upon calculation, the number of DNAs in the sample to be tested in the polymerase chain reaction solution meets expectations. The size of the droplet was 41 μm, and the total number was 4.43×I0⁵ . The number of the DNA molecules added was about 1.26×I0⁴ after being quantified by commercial digital PCR, and about 1.23×I0⁴ fluorescent droplets were obtained in the detection method of the present disclosure, which meets the Poisson distribution expectation.

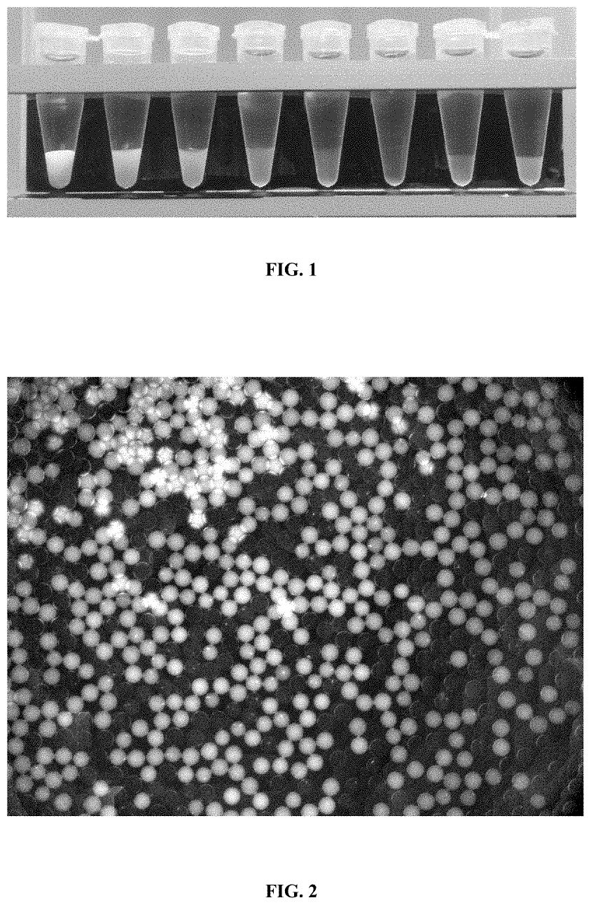

After the thermal cycling reaction was completed, the light sheet scanning imaging method was used for detection, as shown in FIG. 2. Obvious fluorescence enhancement appears in some of the droplets. It can be seen that although the concentration of the betaine solution added as the refractive index enhancer is as high as 3. ISM, polymerase chain reaction still occurs, which indicates that the method for transparentizing the droplets does not cause the reduction of the specificity of the polymerase chain reaction and is capable of enabling DNA detection with single base difference present.

### EXAMPLE 4

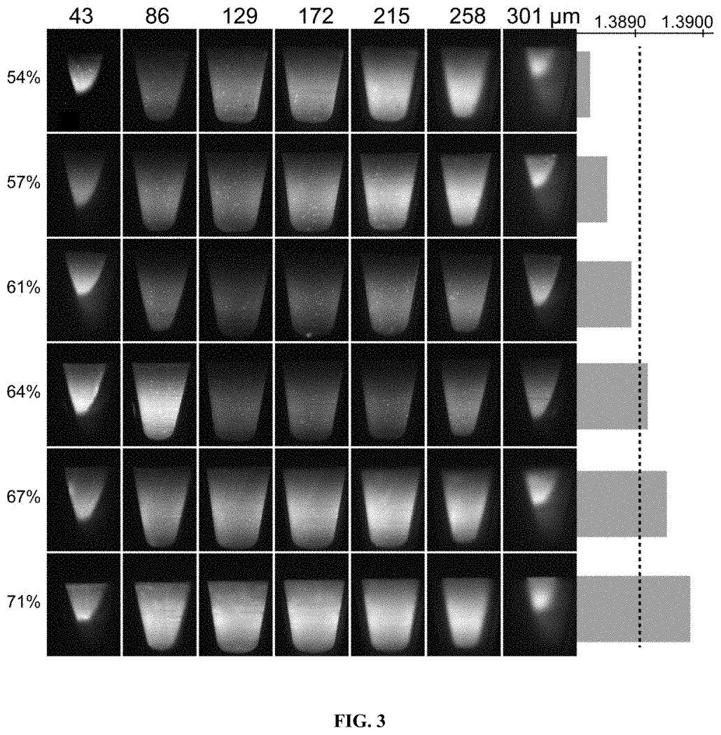

Light sheet scanning was used to image the polymerase chain reaction solution added with different concentrations by volume of 5 mol/L betaine solution, as shown in FIG. 3. The number on the left of FIG. 3 is the ratio of 5 mol/L betaine solution to the total sample solution (i.e., 54%, 57%, 61%, 64%, 67%, 71%, all are volume percentages), and the upper number is the imaging depth (the distance to camera). The addition amount of betaine solution increases from top to bottom, the corresponding refractive index increases. The gray horizontal bar on the right represents the refractive index of the water phase in the sample, and the dotted line on the right is the refractive index of the oil phase. From left to right, the distances between the excitation surface of the light sheet and the droplets in the outermost layer increase sequentially. It can be seen that rows 1, 2, 5, and 6 have insufficient transmission depth due to insufficient refractive index matching, so that the fluorescent bright spots cannot be identified when the imaging depth is less than 200 microns; however, clear fluorescence can still be seen when the imaging depths of rows 3 and 4 are more than 300 microns. It can be seen that the penetration depth is affected when the degree of refractive index matching is different.

### EXAMPLE 5

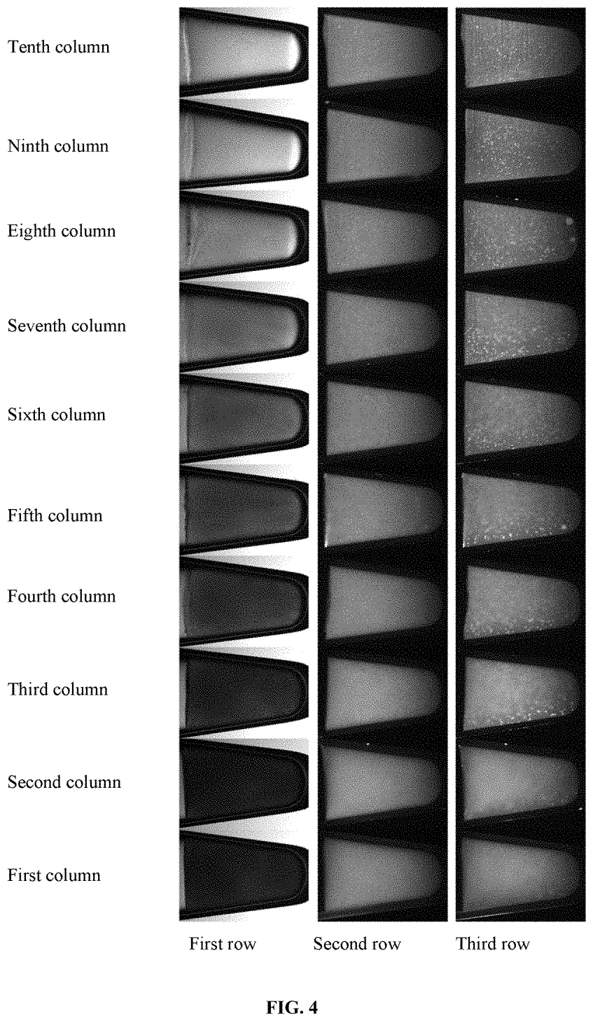

The polymerase chain reaction solutions added with different concentrations by volume of 5 mol/L betaine solution were imaged under several illumination conditions, respectively, as shown in FIG. 4. With a concentration interval of 2%, the ratio of 5 mol/L betaine solution from the first column to the tenth column to the total sample solution is 47%, 49%, 51% . . . 65%. Each column is a different imaging method of the same sample, and the imaging position and the distance of the acquisition device are kept unchanged; wherein the first row is bright-field imaging, the second row is wide-field illumination fluorescence imaging, and the third row is light sheet scanning imaging. It can be seen that the fluorescent signal cannot be obtained in the bright field, while the recognition of the droplets is not facilitated in the wide-field illumination due to the multi-layer signal superposition. For light sheet scanning imaging, when the addition amount of refractive index enhancer is insufficient, the refractive index of the water phase cannot be increased to a degree matched with that of the oil phase, resulting in only droplets in the shallow layer being imaged (the first to the fifth column in the third row). When the refractive index enhancer is added in an amount sufficient to match the refractive index of the water phase with that of the oil phase, the droplets in the shallow layer and deep layer can be imaged at the same time (the sixth to the tenth column in the third row). Samples with matching refractive index have better transparency, so that a clearer fluorescence picture can be obtained (refractive index of the seventh column in the third row is the best match, and the fluorescence picture is the clearest). At the same time, compared to the wide-field illumination, light sheet illumination can avoid the superposition of multi-layer fluorescent signals and obtain internal fluorescent details.

### EXAMPLE 6

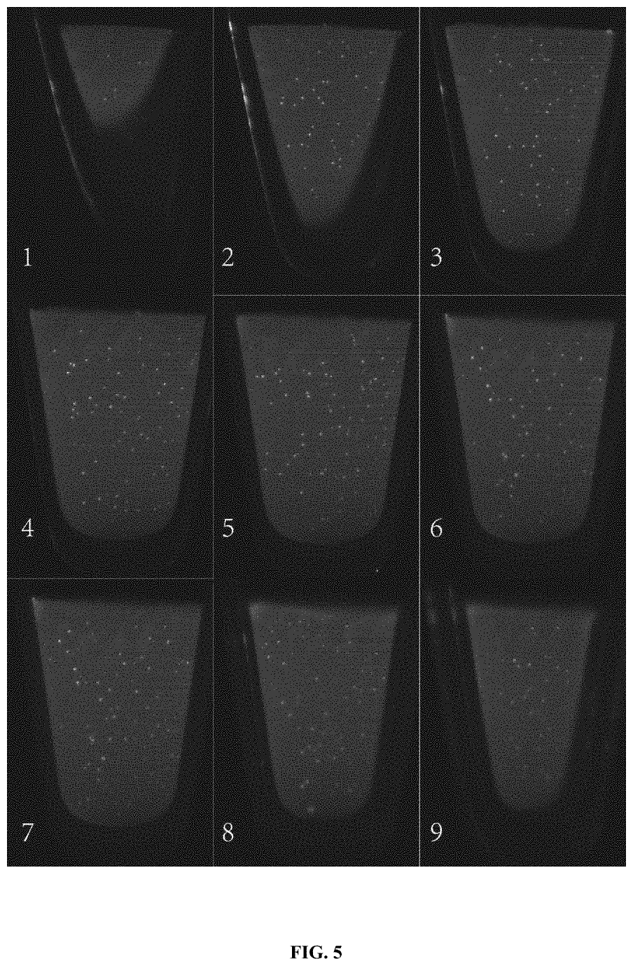

After amplification, the digital PCR transparent macroemulsion was still in the PCR tube, and tomographic imaging was performed on the entire tube by using the light sheet illumination imaging device. FIG. 5 shows the results of imaging the PCR tube at varying depths from the end closest to the imaging end to the end farthest from the imaging end. It can be seen that the fluorescent signal is clearly visible in the droplets from the beginning to the end, the signal-to-noise ratio is high enough for counting.

### EXAMPLE 7

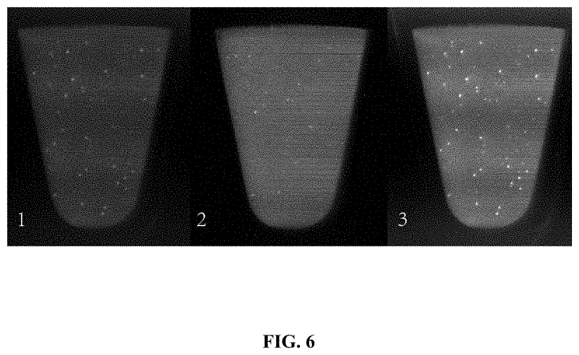

In the PCR Mixed solution in the water phase of the transparent macroemulsion, a pair of primers, two probes that differ by only one base but were fluorescently labeled differently were used to detect single base differences. The DNA samples extracted from heterozygous human peripheral blood were used as the detection objects, different fluorescent signal spot patterns were respectively seen in two different fluorescence channels (FIG. 6-1, and FIG. 6-2), and no obvious signal overlap was seen in the summed image of the two (FIG. 6-3). In this example, it can be seen that this technology has the ability to recognize the difference of a single DNA base.

Furthermore, in addition to silicone-based oil, the oil phase matrix in the oil phase can also be fluorocarbon oil or hydrocarbon-based oil, but the silicone-based oil has stronger capability of dissolving other substances, is easier to adjust the refractive index, and is easier to form a stable emulsion system that can be compatible with the biological reactions.

Those skilled in the art can easily understand that the foregoing contents are only preferred embodiments of the present disclosure and are not intended to limit the present disclosure. Any modifications, equivalent substitutions, improvements, and the like made within the spirit and principle of the present disclosure shall be included into the protection scope of the present disclosure.

What is claimed is:

1.-33. (canceled)

34. A combination reagent for preparing a water-in-oil transparent emulsion, said combination reagent comprising:
a water phase reagent in which a refractive index enhancer is dissolved; and
an oil phase reagent in which a surfactant is dissolved;
wherein when optical detection is performed, an imaging depth of water-in-oil droplets formed by the water phase reagent and the oil phase reagent is at least 300 microns.

35. The combination reagent of claim 34, wherein an absolute value of a difference between refractive indexes of the water phase reagent and the oil phase reagent is not more than 0.1.

36. The combination reagent of claim 34, wherein a mass percentage of the refractive index enhancer in the water phase reagent is not less than 20%.

37. The combination reagent of claim 34, wherein the refractive index enhancer is at least one selected from the group consisting of inorganic salt, monosaccharide, disaccharide, polysaccharide derivative, amino acid, polar organic compound, dimethyl sulfoxide, formamide, tetramethylammonium chloride and bovine serum albumin.

38. The combined reagent of claim 37, wherein the polar organic compound is at least one selected from the group consisting of acetylcholine, choline, betaine, and ceramide.

39. The combination reagent of claim 37, wherein the amino acid is at least one selected from the group consisting of glycine, arginine, threonine, and lysine.

40. The combination reagent of claim 34, wherein the oil phase reagent comprises at least one matrix selected from the group consisting of fluorocarbon oil, hydrocarbon-based oil, silicone-based oil and derivatives thereof.

41. The combination reagent of claim 34, wherein a HBL value of the surfactant is not more than 8.

42. The combination reagent of claim 34, wherein a mass percentage of the surfactant in the oil phase reagent is from 0.1% to 20%.

43. The combination reagent of claim 34, wherein the surfactant comprises a silicone-based surfactant, a fluorocarbon-based surfactant, a hydrocarbon-base surfactant, a polydimethylsiloxane, a polydimethylsiloxane derivative, a polyethylene glycol (PEG), a polypropylene glycol (PPG), or any combination thereof.

44. A transparent emulsion, comprising:
a water phase in which a refractive index enhancer is dissolved;
an oil phase in which a surfactant is dissolved; and
an analyte;
wherein the water phase forms discrete droplets, the oil phase forms a continuous phase, the analyte is in the discrete droplets of the water phase, and when an optical detection is performed, an imaging depth of the droplets is at least 300 microns.

45. The transparent emulsion of claim 44, wherein a volume percentage of the water phase in the transparent emulsion is from 5% to 90%.

46. The transparent emulsion of claim 44, wherein the analyte is selected from nucleic acid, protein, bioactive molecule, bacteria, and cell.

47. A droplet for providing an independent micro-encapsulation environment, wherein said independent micro-encapsulation environment is obtained by emulsifying and dispersing the combination reagent of claim 34.

48. The droplet of claim 47, wherein an average diameter of a water phase droplet in the droplet is not less than 0.2 μm.

49. The droplet of claim 47, wherein the droplet provides an independent micro-encapsulation environment for a digital polymerase chain reaction.

50. Use of the droplet of claim 47 in an in-situ closed imaging detection or a digital polymerase chain reaction.

51. A method of digital polymerase chain reaction, comprising:
dispersing an analyte in a water phase reagent in which a refractive index enhancer is dissolved;
contacting the water phase reagent with the oil phase reagent in which a surfactant is dissolved to form a water-in-oil emulsion comprising droplets, and wherein the analyte is in said droplets;
amplifying the analyte in said droplets; and
performing an optical detection on said droplets.

52. The method of claim 51, wherein an absolute value of a difference between refractive indexes of the water phase reagent and the oil phase reagent is not more than 0.1.

53. The method of claim 51, wherein the amplification is selected from polymerase chain reaction, multiple displacement amplification reaction, recombinase polymerase isothermal amplification reaction, loop-mediated isothermal amplification reaction, or rolling circle amplification reaction.

54. The method of claim 51, wherein the optical detection is selected from light sheet scanning imaging, wide-field scanning imaging, bright-field imaging or confocal imaging.

55. The method of claim 51, wherein the method has a sensitivity of a single base.
